(19) United States
(12) Patent Application Publication
(10) Pub. No.: US 2009/0287170 A1
(43) Pub. Date: Nov. 19, 2009

(54) URINE COLLECTION SYSTEM

(75) Inventor: Edgar A. OTTO, Boca Raton, FL (US)

Correspondence Address: CHRISTOPHER & WEISBERG, P.A., 200 EAST LAS OLAS BOULEVARD, SUITE 2040, FORT LAUDERDALE, FL, 33301, US

(73) Assignee: PREFERRED MEDICAL DEVICES, INC., Boca Raton, FL (US)

(21) Appl. No.: 12/119,779

(22) Filed: May 13, 2008

(51) Int. Cl. A61M 1/00; A61B 5/20

(52) U.S. Cl. 604/318; 604/327; 600/573

(57) ABSTRACT

The present invention provides a system for collecting urine, including a housing defining an interior cavity and a trough extending along a substantial length of an exterior surface thereof; a peristaltic pumping mechanism coupled to the housing; a collection receptacle positionable within the trough of the housing, the collection receptacle defining an opening for receiving urine; an optical sensor proximate the trough to detect a position of the collection receptacle, the sensor being operable to activate the peristaltic pumping mechanism; a reservoir in fluid communication with the collection receptacle; and a conduit providing a fluid path between the collection receptacle and the reservoir, the conduit being coupled to the peristaltic pumping mechanism to facilitate fluid flow from the collection receptacle to the reservoir.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention is generally directed to urine collection devices, and more particularly, to compact, portable urine collection methods and systems for collecting and storing urine from individuals who may be unable to use conventional facilities.

### BACKGROUND OF THE INVENTION

Removal of urine from incapacitated humans has been undertaken using a variety of devices and methods with limited success. For instance, diapers have been used to capture urine from patients. While diapers may collect most of the urine produced by a patient, diapers hold moisture against the skin, which can cause rashes on the patient. In addition, diapers must be changed frequently to function adequately and avoid leakage. The idea of wearing a diaper, having to remain in a soiled diaper, and the difficulties associated with changing and disposing of soiled diapers involve significant psychological, emotional, and logistical drawbacks for users of diapers. For at least these reasons, diapers are not a desirable choice for collecting urine from a patient.

Another device commonly used to collect urine from incapacitated patients who are unable to use conventional toilets is a bedpan. Bedpans have been used successfully but produce undesirable odors and are, at times, unsightly. In addition, bedpans often require the assistance of a nurse to use. While a nurse is respectful of a patient's privacy, nurses often make patients uncomfortable.

Catheters and urine condoms are also commonly used to remove urine from men. Catheters are typically composed of small diameter tubing that is placed inside the urethra of a patient. Urine condoms are worn on the exterior of the penis. While catheters and urine condoms may be efficient at removing urine from men, these devices frequently cause infection and may be painful or uncomfortable to insert or wear.

A class of urine collection devices in addition to those discussed above include urine suction devices, which typically transport urine from a patient to a tank or reservoir using pumps. While such suction collectors overcome some of the disadvantages of the diaper and the bedpan, such suction collectors include a number of drawbacks. For example, suction devices often lack portability, require a vacuum coupling with the skin of the user that must be adjusted, a medical attendant to operate the device, a large separate tank for separating urine from air, and a separate AC powered vacuum source. In addition to these requirements, such devices generally lack safety features that would avoid injury to the users. For example, there may be no mechanism in place to shut down a suction collector in the case of too much vacuum pressure or a filled tank.

In view of the above limitations, it would be desirable to provide an apparatus for removing urine that provides (i) diminished risk of skin irritation, rashes and sores because moisture content near the user is reduced; (ii) reduction in the high incidence of infection and resulting costs of treatment because the device is external and self-applied; (iii) sufficient volume storage for a substantial quantity of urine before discarding (as opposed to other alternative that require immediate disposal); (iv) reduction in the instances of “slip and fall” accidents, as the device will eliminate the necessity of the users to transfer locations or rush to the bathroom; and (v) ease of use and maintenance.

### SUMMARY OF THE INVENTION

The present invention may include a compact urine collection system adapted to receive and reliably store urine from a user. In particular, the present invention provides a system for collecting urine, including a housing defining an interior cavity and a trough extending along a substantial length of an exterior surface thereof; a pumping mechanism coupled to the housing; a collection receptacle positionable within the trough of the housing, the collection receptacle defining an opening for receiving urine; a reservoir in fluid communication with the collection receptacle; and a conduit providing a fluid path between the collection receptacle and the reservoir, the conduit being coupled to the pumping mechanism to facilitate fluid flow from the collection receptacle to the reservoir. The pumping mechanism may include a peristaltic pump, and the pumping mechanism may include a pump housing containing the peristaltic pump therein, the system further having a sensor operable to terminate or prevent operation of the pump based at least in part upon a detected condition of the pump housing. A check valve may be in fluid communication with the collection receptacle to prevent backflow. In addition, the housing may include at least one status indicator providing an indication of a condition of the pumping mechanism, and a sensor may be coupled to the housing to detect a position of the collection receptacle, the sensor being operable to activate and/or deactivate the pumping mechanism. The system of the present invention may further include a urinalysis module coupled to the housing, the urinalysis module operable to analyze a property of urine in at least one of the collection receptacle, conduit, and reservoir.

The present invention also provides a method of collecting urine, including removing a urine collection receptacle from a housing; actuating a pumping mechanism upon detection of the removal of the urine collection receptacle; urinating into the collection receptacle; placing the urine collection receptacle back within proximity to the housing; and terminating operation of the pumping mechanism upon detection of the placement of the urine collection receptacle within proximity to the housing. The detection of the removal and detection of the placement of the urine collection receptacle may include optical detection, and the method may also include analyzing a property of urine in fluid communication with the collection receptacle.

The present invention enables users to urinate in any location with comfort and without assistance, and optional inclusion of a peristaltic pump ensures that the urine collection system is a closed system where every portion of the urine collection device that contacts urine may be easily removed from the device and disposed. Thus, the system is easy to clean. In addition, the urine collection system prevents urine from flowing backwards through the conduit back into the urine collection receptacle after urine has entered the conduit. These and other features and advantages of the present invention will become apparent after review of the following drawings and detailed description of the disclosed embodiments.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a perspective view of an embodiment of a urine collection system constructed in accordance with the principles of the present invention;

FIG. 2 is an additional perspective view of an embodiment of a urine collection system constructed in accordance with the principles of the present invention;

FIG. 3 is a front view of an embodiment of a urine collection system constructed in accordance with the principles of the present invention;

FIG. 4 is a rear view of an embodiment of a urine collection system constructed in accordance with the principles of the present invention;

FIG. 5 is a top view of an embodiment of a urine collection system constructed in accordance with the principles of the present invention;

FIG. 6 is a side view of an embodiment of a urine collection system constructed in accordance with the principles of the present invention; and

FIG. 7 is still another perspective view of an embodiment of a urine collection system constructed in accordance with the principles of the present invention.

### DETAILED DESCRIPTION OF THE INVENTION

The present invention provides a urine collection system for collecting urine from users that is discrete, compact, includes fail-safe safety measures, and is adapted to be used without assistance from health professionals or other caregivers. A urine collection system in accordance with the present invention may be used to collect urine from humans and animals alike. More specifically, the disclosed urine collection system may be used to collect urine from bedridden patients, wheelchair-bound users, users who may have limited mobility, users who have various forms of urinary incontinence, people unable to use conventional toilets, users who are traveling, and others. The present urine collection system may also be used to collect urine from patients in various positions, such as, but not limited to, a seated position, a standing position, a prostrate position, a reclined position, an upright position, and other positions.

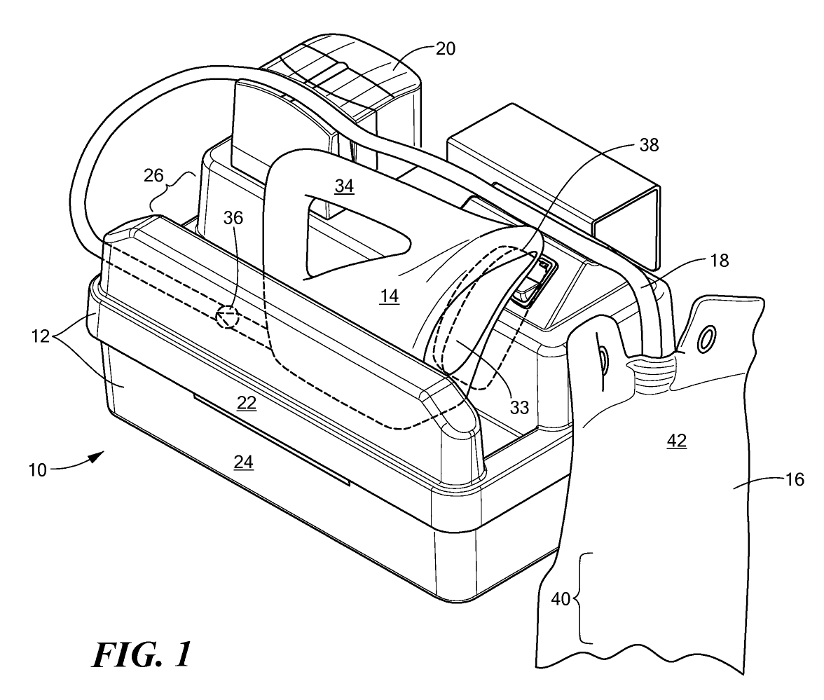

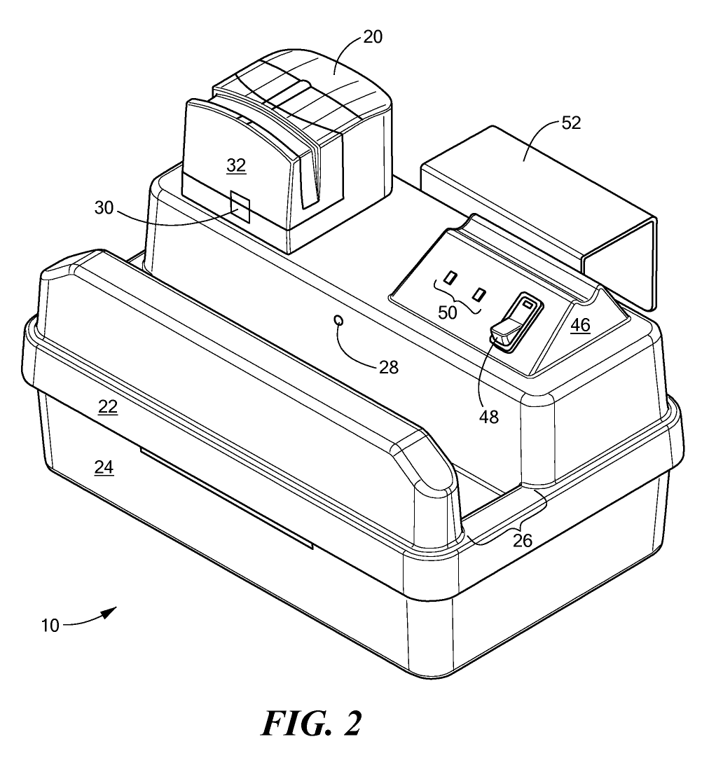

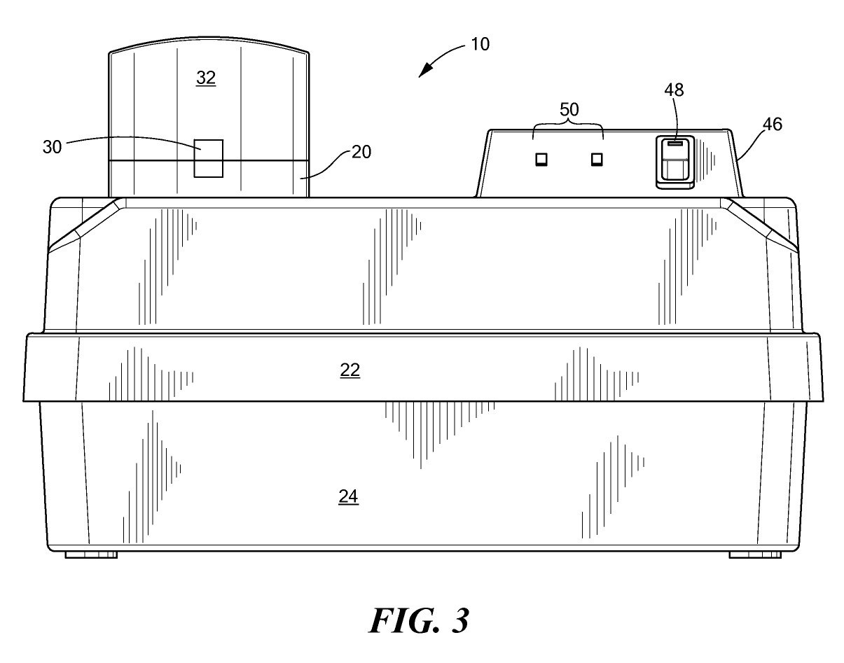

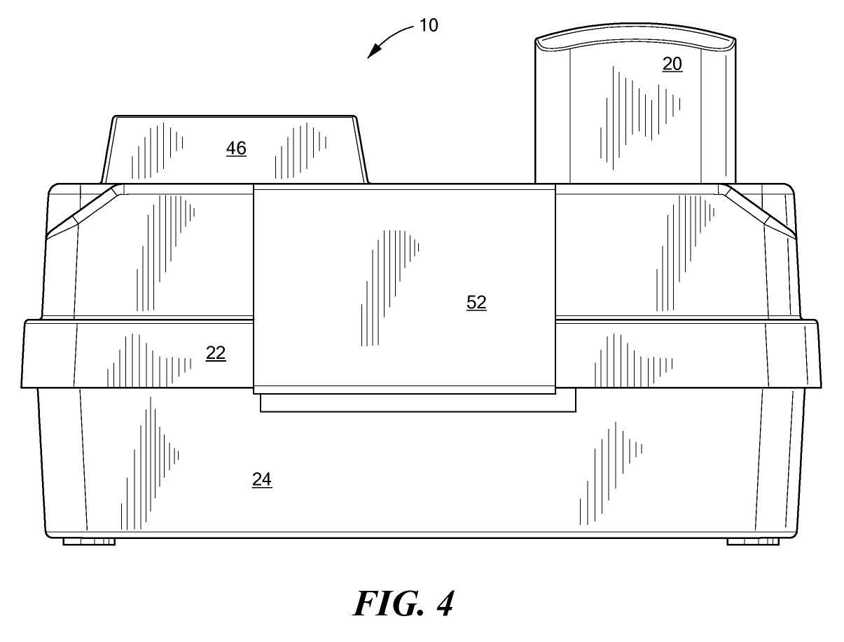

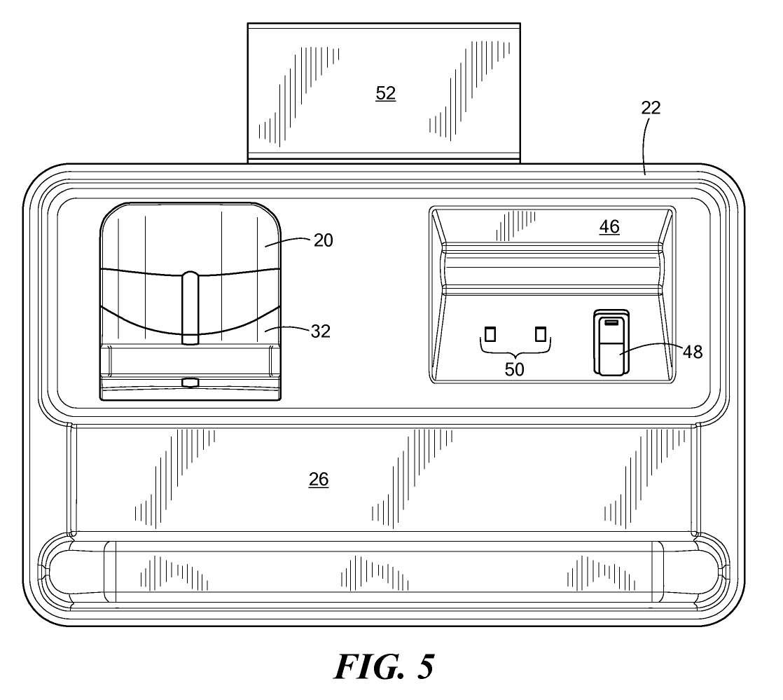

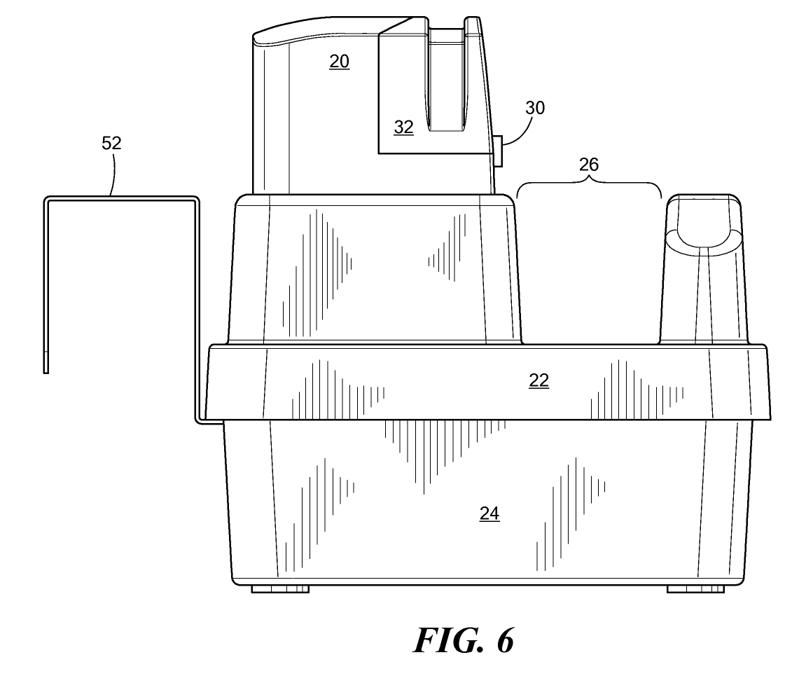

Turning now to the drawings, embodiments of the present invention are described in detail. Now referring to FIGS. 1-6, a urine collection system is shown, generally designated as “10”. The collection system 10 generally includes a housing 12 containing or otherwise enclosing one or more operative components of the system 10, a urine collection receptacle 14 or cup, and a urine collection reservoir 16. The collection receptacle 14 may be in fluid communication with or otherwise coupled to the collection reservoir 16 via a conduit 18, such as tubing or the like, as described in more detail below. Both the collection receptacle 14 and the reservoir 16 may be mounted onto or otherwise positioned about the housing 12, where the housing 12 provides one or more controls or indicators as well as a pumping mechanism 20 employed for operation of the urine collection system 10.

In particular, the housing 12 may include one or more components engageable with or otherwise assembled to one another to define an interior cavity as well as one or more exterior surfaces or locations for placement and connection of the collection receptacle 14 and reservoir 16. For example, the housing 12 may include a first housing component 22 that rests atop or is connected to a second housing component 24. The second housing component 24 may include a hollowed tray or base-type structure, while the first housing component 22 may define a lid-type element positionable over the second housing component 24. When assembled to one another, the first and second housing components define an interior cavity for placement of one or more mechanisms or components of the urine collection system 10.

The first housing component 22 may define a trough 26 extending along a substantial length thereof for receiving the collection receptacle 14. The trough 26 may be open and therefore accessible on either end of the first housing component 22 to allow the receptacle 14 to be removed or replaced ambidextrously via either side of the system 10. In other words, the receptacle 14 is readily positionable within the trough 26 in different orientations (i.e., facing one side, facing the opposite side, etc.) and/or from varying positions with respect to the housing 12 (i.e., whether the user is on the left side of the housing, the right side, directly behind, in front, etc.). In addition, one or more sensors or triggers 28 may be disposed within or otherwise positioned in proximity to the trough 26 to detect the removal or replacement of the receptacle 14 in the trough 26, and to initiate or terminate operation of a pumping mechanism 20. Any number of different sensors may be used, for example, optical sensors, pressure sensors, contact switches, inductive sensors, capacitive sensors, reflective sensors, etc. Moreover, one or more sensors may be used to detect that the reservoir 16 coupled to the collection receptacle 14 is nearly full or has become disconnected from the pumping mechanism 20.

The housing 12 may be manufactured from a wide variety of materials that are easy to clean and generally waterproof or water resistant (such as, for example, plastic, synthetic leather or rubber, vinyl, etc.). In addition to the housing 12, the materials for the component parts of the present invention may be chosen to minimize the overall weight of the system 10 while still providing durability and reliability. For example, light weight, durable plastics may be used as much as possible and aluminum may be used where metal parts are desirable or appropriate.

The pumping mechanism 20 may be disposed on an exterior surface of the first housing component 22, where the pumping mechanism 20 is operably connectable to at least a portion of the collection receptacle 14, conduit 18, and/or reservoir 16 to direct collected urine from the collection cup to the reservoir 16. For example, the pumping mechanism 20 may include a pump housing having a peristaltic pump disposed therein. The peristaltic pump may include one or more rollers coupled to a portion of the conduit 18 connecting the receptacle 14 to the reservoir 16 to create a pressure gradient drawing urine away from the collection receptacle 14, through the conduit 18, and into the reservoir 16. In addition, the collection system 10 may include a safety sensor or switch 30 operable to terminate operation of the pumping mechanism under certain circumstance where continuous operation may pose a safety risk. For example, the pumping mechanism 20 may include a pump housing having a retractable or removable component 32 that can be opened to expose the rollers of the peristaltic pump for insertion of a portion of the conduit 18. Peristaltic pumps operate via movable rollers, which may create a pinch point that could injure a person when exposed. As such, when the retractable component 32 of the pump housing is opened and/or the peristaltic pump rollers are otherwise exposed, the safety sensor 30 may detect the opened condition, and thus prevent or terminate operation of the pump. The sensor 30 may be in communication with a controller having programmable logic stored therein to react to the sensor information, or the sensor 30 may be operable to create an open circuit with a power supply to the pumping mechanism 20, thus preventing operation of the pump, when the rollers are exposed. Once the retractable portion 32 of the pump housing is closed or the rollers are otherwise guarded, the pump may once again become operable.

In a particular example, the pumping mechanism 20 may be calibrated to create a flow rate of approximately 1.2 to approximately 1.4 liters/minute. Higher flow rates (e.g., rates greater than 1.4 liters/minute) may be used to remove urine at an increased rate if necessary. Slower flow rates (e.g., rates less than 1.2 liters per minute) may not sufficiently draw urine out of the collection receptacle 14, thereby possibly resulting in undesired splash-back and overflow. The flow rate may be adjustable by the user through the modification or setting of one or more parameters of the pumping mechanism (i.e., voltage, current, etc.) via a pump control input (not shown), and as such, any rate greater than the rate of urination may be used.

In an alternative configuration, the housing 12 may include a connection point or other interface (not shown) for the system 10 to connect to an existing hospital scavenging or vacuum system. As such, a pressure gradient may be established within the reservoir 16, conduit 18, and or collection receptacle 14 by the vacuum or scavenging system to draw urine flow away from the receptacle 14 and into the reservoir 16 and/or directly into the scavenging system.

Referring to FIG. 1, the collection receptacle 14 may generally have a receptacle body including an opening 33 therein for receiving a fluid, as well as a handle 34 on an exterior portion thereof to aid in handling and manipulating the receptacle 14. One or more portions or surfaces of the receptacle 14 may include an antibacterial layer or similar medicament coating to reduce any buildup of harmful or infectious substances during prolonged use of the system 10. The receptacle body may be contoured and/or concave to ease positioning of the receptacle 14 against an individual for use. In particular, the receptacle body and the opening 33 are adapted to both be positioned against the labium when used by a female and to receive a penis when used by a male. The opening 33 may include a rolled inner lip for improved comfort and use. Although not pictured, in some embodiments, the collection receptacle 14 may include one or more orifices or vents that are positioned to both allow air to freely enter the collection receptacle 14 and to prevent urine from leaking out of the collection receptacle 14. The vents may be of sufficient size to prevent vacuum pressure from building up within the collection receptacle 14, and as such, the collection receptacle 14 does not develop or require a sealed vacuum coupling with the skin of the user.

The collection receptacle 14 may further include an interior chamber defined by one or more sidewalls, where at least one of the sidewalls includes a deep channel (not shown) extending along a length thereof to receive and redirect a urine stream without creating and/or minimizing splash-back. A filter (not shown) may be disposed within the collection receptacle 14 and/or a portion of the conduit 18 to prevent debris, large particles in the urine, and any other foreign matter from entering the conduit 18, reservoir 16, and/or pumping mechanism 20. In addition, a reflux valve 36 may also be disposed within the collection receptacle 14 and/or portion of the conduit 18 to prevent undesirable backward flow of urine from the conduit 18 into the collection receptacle 14.

In addition, the collection receptacle 14 may include an absorbent material, layer or the like 38 that can be applied to the body for prolonged periods of time. For example, should a patient or user be unconscious or unable to manipulate and position the collection receptacle 14, the receptacle 14 may be affixed or otherwise coupled to the patient for a desired period of time. Upon urinating, the absorbent material 38 of the collection receptacle 14 may include sufficient absorbency characteristics to draw the urine away from the patient, and further, the pumping mechanism 20 may be triggered upon a detection of moisture within a portion of the absorbent material 38 to further draw urine into the conduit 18 and reservoir 16, such that the area of skin around the collection receptacle 14 is sufficiently dry within a short period of time for improved comfort and hygiene during prolonged use.

Still referring to FIG. 1, the urine collection reservoir 16 of the present invention may be coupled or otherwise affixed to a portion of the housing, and may further include a first opening in fluid communication with the interior cavity of the reservoir 16, where the first opening may be coupled to the conduit 18 and/or collection receptacle 14 described above for receiving urine. The opening may include an angled connector that may be rotatable and/or able to swivel to ease the coupling of the reservoir 16 to a particular fluid conduit 18. The collection reservoir 16 may further include a check valve coupled to or otherwise in fluid communication with the first opening to ensure that fluid only flows into the reservoir 16 while limiting or preventing backflow out of the first opening.

In addition to the first opening, the collection reservoir 16 may include or define a second opening (not shown) in fluid communication with the interior cavity of the reservoir 16 to facilitate draining or emptying of the reservoir 16 when a particular fluid level within the reservoir 16 has been reached. A particular fluid level may be assessed by including a fluid level indicator 40 on an exterior surface of the reservoir 16, where the fluid level indicator may include a graduated scale of fluid measurements corresponding to a particular fluid level in the reservoir 16 (i.e., ml, oz., etc). The fluid collection reservoir 16 may further define a vent or other opening 42 to allow excess pressure to be released and further reduce the likelihood that the reservoir 16 will rupture or experience a structural failure due to excessive amounts of gas pressure or the like building up in the reservoir 16. The vent 42 may also include a filter that prevents particles of a certain size or the like from escaping the reservoir 16. The reservoir 16 may have sufficient volumetric capacity to hold one or more days worth of urine (e.g., approximately 1 to 2 liters).

The conduit 18 tubing interconnecting the collection receptacle 14 to the reservoir 16 may be a pre-coiled (or a straight) length of about 0.25″ (e.g., approximately 0.125″ to approximately 0.5″) inner diameter tubing manufactured from a durable, clear material. In a particular application, approximately 25″ to approximately 32″ of tubing may be used. Within at least a portion of the conduit 18, a soft, flexible material (e.g., rubber) may be inserted in-line with the conduit 18 that is adapted to absorb percussive forces generated by the pumping mechanism and the fluid flow. This percussion relief tube disposed inline between the conduit 18 and the pump at the pump helps to significantly reduce the operating noise level of the system 10. Moreover, the material used for the percussion relief tube may be selected so as to include a material property such that the tube collapses and thus prevents fluid flow therethrough when a vacuum pressure in the conduit 18 exceeds a desired failsafe threshold, e.g., such as approximately 10 feet of water for example. Thus, if an obstruction occurs in the system 10, a failsafe measure is provided to prevent any potentially dangerous or harmful buildup of vacuum pressure.

Of note, the collection receptacle 14, conduit 18, and reservoir 16 may be sealed or otherwise permanently affixed to one another to provide a closed fluid flow path that is self-contained, and thus readily replaceable or interchangeable. The sealed fluid flow path ensures that the housing 12 of the collection system 10 and/or the pumping mechanism 20 does not come into contact with urine, and is thus not subject to contamination. Such a configuration eliminates the need to sterilize the housing 12 after use, eases overall use of the system 10, and reduces downtime or difficulties in replacing the reservoir 16, cup or the conduit 18. Rather, the sealed collection receptacle 14, conduit 18, and reservoir 16 may be discarded after use, and a new, sterile unit may be replaced for further operation in conjunction with the housing 12 in a short period of time. The components may be glued, welded, or otherwise permanently affixed to one another to provide the desired sealed configuration.

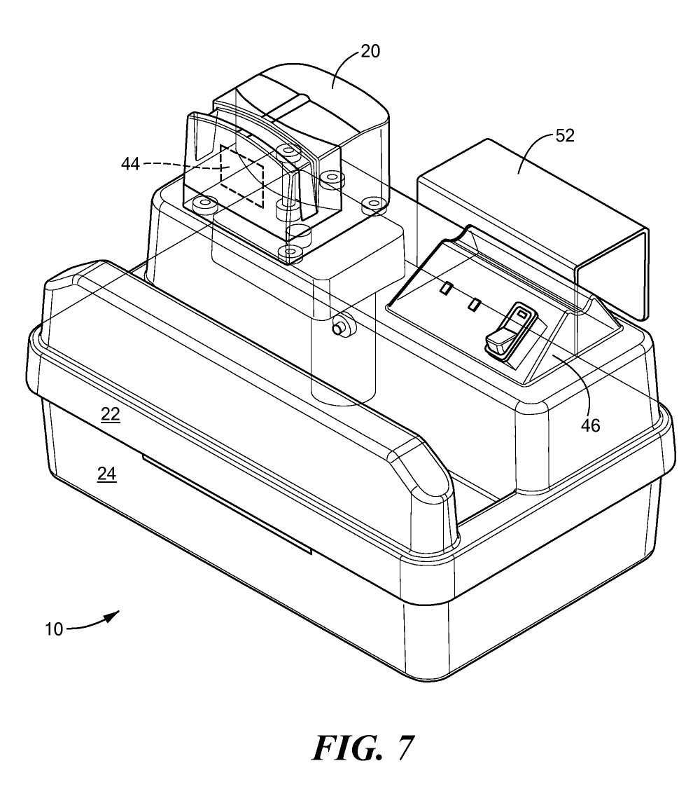

Now referring to FIG. 7, the collection system 10 of the present invention may further include a urinalysis module 44 coupled to or otherwise disposed within a portion of the housing 12 for assessing one or more compositional properties of urine being collected, as well as monitoring the flow rate and thus the overall urine volume voided into the system 10 over a particular period of time. In particular, the urinalysis module 44 may be coupled to a portion of the receptacle 14, conduit 18, and/or reservoir 16 for a determination of the contents of the collected urine, which may then be stored, transmitted, and/or otherwise accessible as needed during patient-monitoring for diagnostic or other healthcare-related issues. The urinalysis module 44 may include an optical sensor as well as a reflector or refractory surface disposed about a portion of the fluid flow path defined by the receptacle 14, conduit 18, and reservoir 16 to perform compositional and volumetric/flow rate measurements. For example, the sensor and/or reflector may be positioned within the pump housing on an upper or top surface of the housing 12. The urinalysis module 44 may also include one or more hardware and/or software components to process, analyze, store, or otherwise manipulate the information provided by the sensor to produce usable, readable results pertaining to the properties or contents of the urine interacting with the sensor. For example, the collection system 10 may include an electronic storage element disposed within the housing 12 for storing, recording, and/or otherwise maintaining information obtained from the urinalysis module. The system 10 may also include a communication interface or component (such as a USB, Ethernet or wireless component) to allow the data to be accessed and/or transferred to secondary electronic devices for further use.

In a particular use, the urine can be analyzed by the module to determine the amount of protein, glucose, blood, etc. in the urine based on their correlating optical properties, as is known in the art. Further, as urine flows through the system 10, the flow rate may be monitored, and thus the volume can be deduced. Moreover, the system 10 may trigger an alarm or provide other indication that the total collected urine volume is close to exceeding the reservoir capacity based on the volume calculation.

In addition to the urinalysis module 44, the urine collection system 10 of the present invention may include a component identification and/or patient tracking module (not shown) to correlate information obtained by the urinalysis module and/or to correlate one or more components of the system 10 (such as the collection receptacle 14/conduit 18/reservoir 16 unit) to a patient. For example, each reservoir 16/conduit 18/receptacle 14 unit that is used with the system 10 over a period of time for a particular patient may be identified and/or tracked to associate the system components with that particular patient. In particular, the reservoir 16/conduit 18/receptacle 14 units may each include a unique identifying component, such as an RFID, bar code or the like, that allows the units to be tracked or otherwise monitored in correlation with a particular set of patient records. As such, when the units are interchanged, as described above, the total number of units, and thus the amount of urine or the like discharged by a patient can be recorded. Such unique identification could also be monitored and/or stored to prevent re-use or recycling of used receptacle 14/conduit 18/reservoir 16 units.

The urine collection system 10 of the present invention may include a power source for operation of the one or more components described herein. In particular, the system 10 may include a portable and/or rechargeable power supply such as one or more batteries, and may also and/or alternatively include the requisite hardware for operation via a typical wall outlet.

Again referring to FIGS. 1-6, the housing 12 may include a surface for a status/control panel 46 on an exterior portion of the first housing 12 component. The status/control panel may provide easy, ergonomic access to, and a view of, the panel by a user seated or positioned adjacent the urine collection system 10. The status/control panel may include a plurality of input elements 48 (i.e., switches, buttons, etc.) to control or otherwise operate a component of the system 10, as well as one or more status indicators 50 that provide a visual, audio, or tactile indication of a condition of one or more components of the collection system 10. In particular, the status indicators 50 may convey information related to the pumping mechanism (such as ON or OFF), a level of fluid within the reservoir 16, information from the urinalysis module 44, etc. The status indicators 50 and/or input elements 48 may be coupled to or otherwise in communication with the components and one or more sensors described herein.

In a particular embodiment, the status indicators 50 may include a number of lights that indicate a condition of the urine collection system 10. For example, when lit, a light labeled “AUTO” may indicate that the urine collection system 10 is in an automated operating mode wherein the pump will turn on when, for example, the collection receptacle 14 is removed from the trough 26 and wherein the pump will turn off when, for example, the collection receptacle 14 is returned to the trough 26. When lit, a light labeled “MANUAL” may indicate that the urine collection system 10 is in a manual operating mode wherein the pump will only turn on if a particular button or switch is activated and wherein the pump will turn off if the button or switch is pressed again. In typical operation, only one of the AUTO and MANUAL lights may be lit at a time. When lit, the light labeled “PUMP” indicates that the pump is activated. When not lit, the PUMP light indicates that the pump is not active. Alternatively and/or in addition, a blinking or flashing PUMP light may indicate that a maximum or threshold run-time of the pump has been exceeded, and as a result, the pump has been turned off automatically.

The system 10 of the present invention may further include a mounting element or bracket 52 coupled to a portion of the housing 12 adapted to securely hold or clamp the system 10 in a convenient location and height. For example, the mounting element 52 may include extended members that fit under a mattress and/or box spring such that the housing 12 is securely held in place by the weight of the mattress and/or box spring. Alternatively, the mounting element 52 may include a hook or substantially “U”-shaped portion for coupling to a particular desired surface or support fixture. In addition, the unit can be attached to a rolling IV cart or other auxiliary medical device platform, such as those commonly found in healthcare facilities.

The system of the present invention was designed as a simple alternative to the in-dwelling catheter, condom catheter, diaper and bed pan. The methods and systems of the present invention are particularly well suited for use by patients with limited mobility, post-operative surgery patients, the elderly (e.g., people ages 65 and over, the Medicare population with “urge incontinence” needs), spinal cord injury (“SCI”) patients; and disabled/chronic condition patients who are non- or partially ambulatory. The present invention also provides a simple and cost-effective solution for UI suffers who do not require internal catheterization.

The present invention offers several additional advantages over prior art devices, including (i) diminished risk of skin irritation, rashes and sores because moisture content near the user is reduced; (ii) reduction in the high incidence of infection and resulting costs of treatment because the device is external and self-applied; (iii) sufficient volume storage (as opposed to other alternative that require immediate disposal); (iv) reduction in the instances of “slip and fall” accidents, as the device will eliminate the necessity of the users to transfer locations or rush to the bathroom; and (v) ease of use and maintenance compared to other prior art devices.

High-level clinical benefits of the present invention include a lower risk of bladder cancer and infections in SCI patients due to the system being an external, self-applied apparatus rather than an inserted catheter. Further, the present invention provides a decreased risk of accidental slips and falls by patients who may be physically unstable or medicated but will not wait for a caretaker to assist with going to the restroom. In addition, many incontinent patients experience continuing exposure to spilled urine increased risk of skin irritation, rashes, and sores. The system of the present invention is operative to draw urine from the patient, thereby decreasing these conditions. Further, prior art urine collection devices overwhelmingly require techniques or assistance typically by trained healthcare professionals. The urine collection system of the present invention empowers non-ambulatory and disabled patients (e.g., both male and female) to be self-sufficient due to the system's simplicity, portability, non-invasiveness, and discrete design.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A system for collecting urine comprising:
a housing defining an interior cavity and a trough extending along a substantial length of an exterior surface thereof;
a pumping mechanism coupled to the housing;
a collection receptacle positionable within the trough of the housing, the collection receptacle defining an opening for receiving urine;
a reservoir in fluid communication with the collection receptacle; and
a conduit providing a fluid path between the collection receptacle and the reservoir, the conduit being coupled to the pumping mechanism to facilitate fluid flow from the collection receptacle to the reservoir.

2. The system according to claim 1, wherein the pumping mechanism includes a peristaltic pump.

3. The system according to claim 2, wherein the pumping mechanism includes a pump housing containing the peristaltic pump therein, the system further comprising a sensor operable to terminate or prevent operation of the pump based at least in part upon a detected condition of the pump housing.

4. The system according to claim 1, further comprising a check valve in fluid communication with the collection receptacle.

5. The system according to claim 1, wherein the housing includes at least one status indicator providing an indication of a condition of the pumping mechanism.

6. The system according to claim 1, wherein the collection receptacle defines a handle to facilitate use thereof.

7. The system according to claim 1, further comprising a sensor coupled to the housing to detect a position of the collection receptacle, the sensor being operable to activate the pumping mechanism.

8. The system according to claim 7, wherein the sensor is an optical sensor located in proximity to the trough.

9. The system according to claim 1, further comprising a urinalysis module coupled to the housing, the urinalysis module operable to analyze a property of urine in at least one of the collection receptacle, conduit, and reservoir.

10. The system according to claim 9, wherein the urinalysis module optically analyzes a compositional property of the urine.

11. The system according to claim 1, further comprising a mounting element coupled to the housing.

12. A system for collecting urine comprising:
a housing defining an interior cavity and a trough extending along a substantial length of an exterior surface thereof;
a peristaltic pumping mechanism coupled to the housing;
a collection receptacle positionable within the trough of the housing, the collection receptacle defining an opening for receiving urine;
an optical sensor proximate the trough to detect a position of the collection receptacle, the sensor being operable to activate the peristaltic pumping mechanism;
a reservoir in fluid communication with the collection receptacle; and
a conduit providing a fluid path between the collection receptacle and the reservoir, the conduit being coupled to the peristaltic pumping mechanism to facilitate fluid flow from the collection receptacle to the reservoir.

13. The system according to claim 12, further comprising a check valve in fluid communication with the collection receptacle.

14. The system according to claim 12, wherein the housing includes at least one status indicator providing an indication of a condition of the pumping mechanism.

15. The system according to claim 12, further comprising a urinalysis module coupled to the housing, the urinalysis module operable to optically analyze a property of urine in at least one of the collection receptacle, conduit, and reservoir.

16. The system according to claim 12, further comprising a mounting element coupled to the housing.

17. The system according to claim 12, further comprising:
a pump housing containing the peristaltic pump therein; and
a sensor operable to terminate or prevent operation of the pump based at least in part upon a detected condition of the pump housing.

18. A method of collecting urine, comprising:
removing a urine collection receptacle from a housing;
actuating a pumping mechanism upon detection of the removal of the urine collection receptacle;
urinating into the collection receptacle;
placing the urine collection receptacle back within proximity to the housing; and
terminating operation of the pumping mechanism upon detection of the placement of the urine collection receptacle within proximity to the housing.

19. The method according to claim 18, wherein detection of the removal and detection of the placement of the urine collection receptacle includes optical detection.

20. The method according to claim 18, further comprising analyzing a property of urine in fluid communication with the collection receptacle.
